(19) United States
(12) Patent Application Publication
(10) Pub. No.: US 2026/0007877 A1
(43) Pub. Date: Jan. 8, 2026

(54) NON-SURGICAL NEURAL STIMULATION SYSTEM BASED ON IN-VIVO SELF-ASSEMBLY

(71) Applicants: UIF (University Industry Foundation), Yonsei University, Seoul (KR); CHUNGANG UNIVERSITY INDUSTRY ACADEMIC COOPERATION FOUNDATION, Seoul (KR); RESEARCH & BUSINESS FOUNDATION SUNGKYUNKWAN UNIVERSITY, Suwon-si (KR)

(72) Inventors: Sang Woo KIM, Seoul (KR); Dong Min LEE, Seoul (KR); Yong Hyun KWON, Goyang-si (KR); Young Min BYUN, Seoul (KR); Han Jun RYU, Pyeongtaek-si (KR); Jang Mook JEONG, Suwon-si (KR)

(73) Assignees: UIF (University Industry Foundation), Yonsei University, Seoul (KR); CHUNGANG UNIVERSITY INDUSTRY ACADEMIC COOPERATION FOUNDATION, Seoul (KR); RESEARCH & BUSINESS FOUNDATION SUNGKYUNKWAN UNIVERSITY, Suwon-si (KR)

(21) Appl. No.: 19/262,700

(22) Filed: Jul. 8, 2025

(30) Foreign Application Priority Data
Jul. 8, 2024 (KR) 10-2024-0089674

(51) Int. Cl. A61N 1/05; A61N 1/36

(52) U.S. Cl. CPC A61N 1/0526; A61N 1/36103; A61N 1/36139

(57) ABSTRACT

The non-surgical neural stimulation includes an electrode layer formed by injecting a first precursor for forming the electrode layer onto a target nerve or into an area therearound and performing a first light exposure process on the first precursor using a first photomask; and an energy harvesting layer formed by injecting a second precursor for forming the energy harvesting layer onto the electrode layer and performing a second light exposure process on the second precursor using a second photomask.

### CROSS-REFERENCE TO RELATED APPLICATION

This application claims priority from Korean Patent Application No. 10-2024-0089674 filed on Jul. 8, 2024 in the Korean Intellectual Property Office, and all the benefits accruing therefrom under 35 U.S.C. 119, the contents of which in its entirety are herein incorporated by reference.

### BACKGROUND

### Field

The present disclosure relates to a non-surgical neural stimulation system based on in-vivo self-assembly, and also relates to a method for manufacturing the non-surgical neural stimulation system based on in-vivo self-assembly.

### Description of Related Art

Unlike conventional medicines that control biological functions using chemical methods, an in-vivo implantable neurostimulation system directly stimulates a target nerve bundle in a pin-point stimulation manner using electrical signals and stimuli, and thus may be applied to nerve regeneration that may be treated in the short term without side effects and various neuropathies, such as, from depression to chronic pain requiring long-term treatment, and Parkinson's disease.

However, in general, a neural stimulation system requires a switching circuit capable of stimulating various modulated electrical signals in order to overcome a neural learning effect on the electrical stimulation. Further, a communication system for identifying a time point at which the electrical signal switching is required is also required. In this switching circuit and communication system, device insertion surgery and removal surgery are essential, and due to the risk of biological side effects such as pathogen infection and immune response that may occur during a surgical operation, numerous patients may have have physical/mental burden.

In order to eliminate the need for the device removal surgery, a neurostimulation system incorporating a biodegradable material is being actively studied around the world. However, the device insertion surgery is still required in this system, and there is a risk of biological side effects caused by the surgical surgery.

### SUMMARY

A purpose of the present disclosure is to provide a neuropathy treatment system for performing an entire process of neuropathy treatment in a non-surgical manner, wherein the process includes manufacturing the system without a surgical operation using a biodegradable precursor that forms a crosslink via light stimulation, and nerve regeneration and treatment via electrical stimulation using the system, and removal of the system.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides a non-surgical neural stimulation system based on in-vivo self-assembly, the system comprising: an electrode layer formed by injecting a first precursor for forming the electrode layer onto a target nerve or into an area therearound and performing a first light exposure process on the first precursor using a first photomask; and an energy harvesting layer formed by injecting a second precursor for forming the energy harvesting layer onto the electrode layer and performing a second light exposure process on the second precursor using a second photomask.

In accordance with some embodiments of the non-surgical neural stimulation system based on in-vivo self-assembly, the first precursor for forming the electrode layer includes: a photocurable polymer monomer; a core-shell particle including an upconversion nanoparticle as a core and an ultraviolet curing agent as a shell; and a conductive nanoparticle.

In accordance with some embodiments of the non-surgical neural stimulation system based on in-vivo self-assembly, the conductive nanoparticle is embedded in the electrode layer.

In accordance with some embodiments of the non-surgical neural stimulation system based on in-vivo self-assembly, the second precursor for forming the energy harvesting layer includes: a photocurable polymer monomer; a core-shell particle including an upconversion nanoparticle as a core and an ultraviolet curing agent as a shell; and a material for forming a piezoelectric layer.

In accordance with some embodiments of the non-surgical neural stimulation system based on in-vivo self-assembly, the material for forming the piezoelectric layer includes a piezoelectric material or an ionic material.

In accordance with some embodiments of the non-surgical neural stimulation system based on in-vivo self-assembly, the piezoelectric material or the ionic material is embedded in the energy harvesting layer.

In accordance with some embodiments of the non-surgical neural stimulation system based on in-vivo self-assembly, each of the first and second photomasks enables negative photo-lithography.

A second aspect of the present disclosure provides a method for manufacturing a non-surgical neural stimulation system based on in-vivo self-assembly, the method comprising: injecting a first precursor for forming an electrode layer onto a target nerve or into an area therearound; placing a first photomask for formation of a target electrode layer structure on a skin; performing a first light exposure process on the first precursor using the first photomask, thereby forming the electrode layer having the target electrode layer structure; injecting a second precursor for forming an energy harvesting layer onto the formed electrode layer; placing a second photomask for formation of a target energy harvesting layer pattern on the skin; and performing a second light exposure process on the second precursor using the second photomask, thereby forming the energy harvesting layer having the target energy harvesting layer pattern.

In accordance with some embodiments of the method for manufacturing the stimulation system based on in-vivo self-assembly, the first precursor for forming the electrode layer includes: a photocurable polymer monomer; a core-shell particle including an upconversion nanoparticle as a core and an ultraviolet curing agent as a shell; and a conductive nanoparticle.

In accordance with some embodiments of the method for manufacturing the stimulation system based on in-vivo self-assembly, the conductive nanoparticle is embedded in the electrode layer.

In accordance with some embodiments of the method for manufacturing the stimulation system based on in-vivo self-assembly, the second precursor for forming the energy harvesting layer includes: a photocurable polymer monomer; a core-shell particle including an upconversion nanoparticle as a core and an ultraviolet curing agent as a shell; and a material for forming a piezoelectric layer.

In accordance with some embodiments of the method for manufacturing the stimulation system based on in-vivo self-assembly, the material for forming the piezoelectric layer includes a piezoelectric material or an ionic material.

In accordance with some embodiments of the method for manufacturing the stimulation system based on in-vivo self-assembly, the piezoelectric material or the ionic material is embedded in the energy harvesting layer.

In accordance with some embodiments of the method for manufacturing the stimulation system based on in-vivo self-assembly, each of the first and second photomasks enables negative photo-lithography.

According to the present disclosure, the neurostimulation system based on in-vivo self-assembly and self-disassembly is provided and is a first system over the world. It is expected that using the present neurostimulation system, the neurostimulation treatment may be realized without the surgical operation in the entire clinical process. Thus, the risk of biological side effects such as pathogen infection due to the surgical operation may be fundamentally eliminated, and the in-vivo self-assembly and self-disassembly may be controlled based on the type and treatment period of a treatment target neuropathy. This is expected to have a significant effect on the field of neurotherapy and regeneration.

The effects of the present disclosure are not limited to the above-mentioned effects, and other effects not mentioned may be clearly understood by those skilled in the art from the following descriptions.

In addition to the above-described effects, the specific effects of the present disclosure will be described together while describing specific matters for implementing the embodiments of the present disclosure below.

### BRIEF DESCRIPTION OF THE DRAWINGS

FIG. 1 is a flowchart illustrating a method for manufacturing a non-surgical neural stimulation system based on in-vivo self-assembly according to an embodiment of the present disclosure.

FIG. 2 illustrates a schematic diagram of a method for manufacturing a non-surgical neural stimulation system based on in-vivo self-assembly according to an embodiment of the present disclosure.

FIG. 3 shows a result of measuring in-vivo energy generation in response to external mechanical stimulation to the system according to an embodiment of the present disclosure.

FIG. 4 is a diagram observing in-vivo energy generation and electric field generation via external mechanical stimulation.

FIG. 5 is a diagram checking biodegradability of a system.

### DETAILED DESCRIPTIONS

Advantages and Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality.

Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and the present disclosure is not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when an element or layer is referred to as being “connected to”, or “coupled to” another element or layer, it may be directly connected to, or coupled to the other element or layer, or one or more intervening elements or layers may be present therebetween. In addition, it will also be understood that when an element or layer is referred to as being “between” two elements or layers, it may be the only element or layer between the two elements or layers, or one or more intervening elements or layers may also be present.

Further, as used herein, when a layer, film, area, plate, or the like is disposed “on” or “on top” of another layer, film, area, plate, or the like, the former may directly contact the latter or still another layer, film, area, plate, or the like may be disposed between the former and the latter. As used herein, when a layer, film, area, plate, or the like is directly disposed “on” or “on top” of another layer, film, area, plate, or the like, the former directly contacts the latter and still another layer, film, area, plate, or the like is not disposed between the former and the latter. Further, as used herein, when a layer, film, area, plate, or the like is disposed “beneath” or “under” another layer, film, area, plate, or the like, the former may directly contact the latter or still another layer, film, area, plate, or the like may be disposed between the former and the latter. As used herein, when a layer, film, area, plate, or the like is directly disposed “beneath” or “under” another layer, film, area, plate, or the like, the former directly contacts the latter and still another layer, film, area, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

When an embodiment may be implemented differently, functions or operations specified within a specific block may be performed in a different order from an order specified in a flowchart. For example, two consecutive blocks may actually be performed substantially simultaneously, or the blocks may be performed in a reverse order depending on related functions or operations.

The features of the various embodiments of the present disclosure may be partially or entirely combined with each other, and may be technically associated with each other or operate with each other. The embodiments may be implemented independently of each other and may be implemented together in an association relationship.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

Further, the term ‘or’ means ‘inclusive or’ rather than ‘exclusive or’. That is, unless otherwise stated or clear from the context, the expression that ‘x uses a or b’ means any one of natural inclusive permutations.

The terms used in the description below have been selected as being general and universal in the related technical field. However, there may be other terms than the terms depending on the development and/or change of technology, convention, preference of technicians, etc. Therefore, the terms used in the description below should not be understood as limiting technical ideas, but should be understood as examples of the terms for illustrating embodiments.

Further, in a specific case, a term may be arbitrarily selected by the applicant, and in this case, the detailed meaning thereof will be described in a corresponding description period. Therefore, the terms used in the description below should be understood based on not simply the name of the terms, but the meaning of the terms and the contents throughout the Detailed Descriptions.

It will be understood that, although the terms “first”, “second”, “third”, and so on may be used herein to describe various elements, components, areas, layers and/or periods, these elements, components, areas, layers and/or periods should not be limited by these terms. These terms are used to distinguish one element, component, area, layer or section from another element, component, area, layer or period. Thus, a first element, component, area, layer or section as described under could be termed a second element, component, area, layer or period, without departing from the spirit and scope of the present disclosure.

The present disclosure relates to a non-surgical neural stimulation system based on in-vivo self-assembly, and also relates to a method for manufacturing the non-surgical neural stimulation system based on in-vivo self-assembly. Specifically, the system of the present disclosure is a non-surgical neural stimulation system based on in-vivo self-assembly and self-disassembly.

The present disclosure provides an in-vivo self-assembly system obtained by injecting a precursor composed of a photocurable polymer monomer having biodegradation characteristics and an IR (infrared) curing photoinitiator onto a target nerve in a pin-point manner and performing a photo-stimulus (IR)-based negative photo-lithography technique on the precursor to precisely control a resulting structure.

A first aspect of the present disclosure provides a non-surgical neural stimulation system based on in-vivo self-assembly, the system comprising: an electrode layer formed by injecting a first precursor for forming the electrode layer onto a target nerve or into an area therearound and performing a first light exposure process on the first precursor using a first photomask; and an energy harvesting layer formed by injecting a second precursor for forming the energy harvesting layer onto the electrode layer and performing a second light exposure process on the second precursor using a second photomask.

The electrode layer may be formed on or around the target nerve. For this purpose, the first precursor for forming the electrode layer is injected onto the target nerve or into the area around the target nerve and performing the first light exposure process on the first precursor using the first photomask to form the electrode layer.

In accordance with some embodiments of the non-surgical neural stimulation system based on in-vivo self-assembly, the first precursor for forming the electrode layer includes: a photocurable polymer monomer; a core-shell particle including an upconversion nanoparticle as a core and an ultraviolet curing agent as a shell; and a conductive nanoparticle.

The photocurable polymer monomer is a polymer which has biodegradation properties, and undergoes a radical reaction by the photo curing photoinitiator and thus is polymerized. Examples thereof include polyethylene glycol diacrylate (PEGDA) and GelMA (Gelatin methacryloyl).

In the core-shell particle, the upconversion nanoparticle is used as the core, and the ultraviolet curing agent is used as the shell.

The photocurable polymer monomer is polymerized based on radicals generated only when a light source having about energy corresponding to the UV energy is irradiated thereto. In this regard, UCNP (the upconversion nanoparticle) is a special nanoparticle for converting a low energy photon into a high energy photon, and examples of the UCNP include NaYF₄:Yb,Tm/NaYF₄, NaYF₄:Yb, Er/NaGdF₄, etc.

The ultraviolet curing agent receives the high energy photons emitted from the UCNP and promotes radical polymerization of the photocurable polymer monomers. The ultraviolet curing agent acts as the shell of the core-shell structure while the UNCP acts as the core thereof. The UCNP has a positive (+) surface charge and the UV curing agent has a negative (−) surface charge. Thus, the UNCP and the UV curing agent may constitute the core-shell structure based on the Coulomb force. Examples of the UV curing agent may include Lithium phenyl (2,4,6-trimethylbenzoyl)phosphinate (LAP), Irgacure 2959, etc.

The conductive nanoparticle is a conductive nanofiller and may include metal nanoparticles, metal nanowires, metal flakes, carbon, etc. The conductive nanoparticles are embedded in the electrode layer.

The energy harvesting layer is formed on the electrode layer. In this regard, the energy harvesting layer may be formed by injecting the second precursor for forming the energy harvesting layer onto the electrode layer and performing the second light exposure process on the second precursor using the second photomask.

In accordance with some embodiments of the non-surgical neural stimulation system based on in-vivo self-assembly, the second precursor for forming the energy harvesting layer includes: a photocurable polymer monomer; a core-shell particle including an upconversion nanoparticle as a core and an ultraviolet curing agent as a shell; and a material for forming a piezoelectric layer.

The photocurable polymer monomer is a polymer which has biodegradation properties, and undergoes a radical reaction by the photo curing photoinitiator and thus is polymerized. Examples thereof include polyethylene glycol diacrylate (PEGDA) and GelMA (Gelatin methacryloyl).

In the core-shell particle, the upconversion nanoparticle is used as the core, and the ultraviolet curing agent is used as the shell.

The photocurable polymer monomer is polymerized based on radicals generated only when a light source having about energy corresponding to the UV energy is irradiated thereto. In this regard, UCNP (the upconversion nanoparticle) is a special nanoparticle for converting a low energy photon into a high energy photon, and examples of the UCNP include NaYF₄:Yb,Tm/NaYF₄, NaYF₄:Yb, Er/NaGdF₄, etc.

The ultraviolet curing agent receives the high energy photons emitted from the UCNP and promotes radical polymerization of the photocurable polymer monomers. The ultraviolet curing agent acts as the shell of the core-shell structure while the UNCP acts as the core thereof. The UCNP has a positive (+) surface charge and the UV curing agent has a negative (−) surface charge. Thus, the UNCP and the UV curing agent may constitute the core-shell structure based on the Coulomb force. Examples of the UV curing agent may include Lithium phenyl (2,4,6-trimethylbenzoyl)phosphinate (LAP), Irgacure 2959, etc.

In accordance with some embodiments of the non-surgical neural stimulation system based on in-vivo self-assembly, the material for forming the piezoelectric layer includes a piezoelectric material or an ionic material.

In accordance with some embodiments of the non-surgical neural stimulation system based on in-vivo self-assembly, the piezoelectric material or the ionic material is embedded in the energy harvesting layer.

In one example, the piezoelectric material may include barium titanate, zinc oxide, or the like. In one example, the ionic material may be NaCl, KCl, ionic liquid, or the like. The piezoelectric material or the ionic material (piezo ionic method) is embedded in the energy harvesting layer.

The formation of the electrode layer or the formation of the energy harvesting layer includes placing the photomask and exposing the light thereto. In this case, a negative photo-lithography is performed using the photomask. The exposure process using the photomask will be further described in detail below.

Hereinafter, a method for manufacturing a non-surgical neural stimulation system based on in-vivo self-assembly according to an embodiment of the present disclosure will be described. Redundant descriptions duplicate with those set forth above will be omitted.

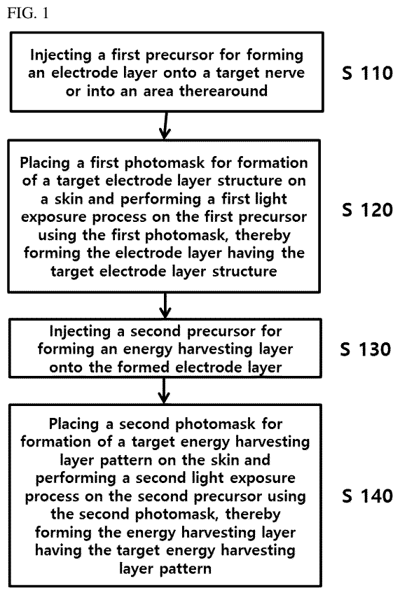

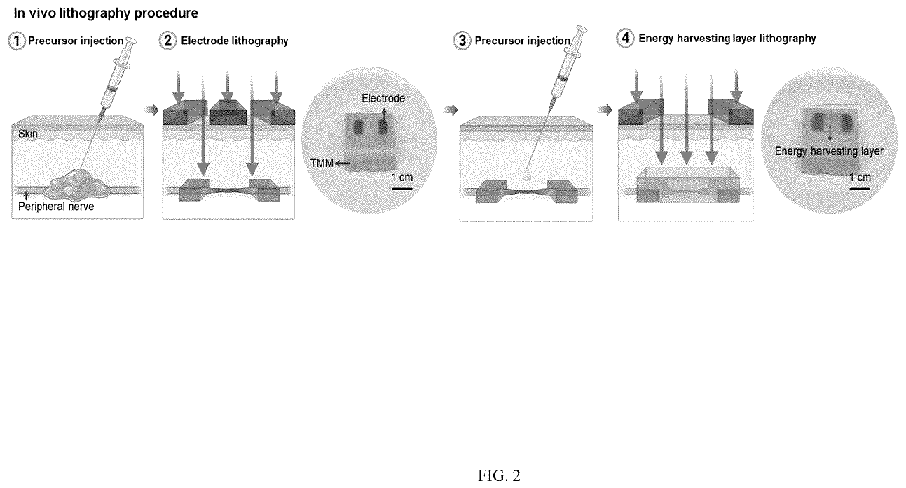

FIG. 1 is a flowchart of a method for manufacturing a non-surgical neural stimulation system based on in-vivo self-assembly according to an embodiment of the present disclosure, and FIG. 2 is a schematic diagram of a method for manufacturing a non-surgical neural stimulation system based on in-vivo self-assembly according to an embodiment of the present disclosure.

A second aspect of the present disclosure provides a method for manufacturing a non-surgical neural stimulation system based on in-vivo self-assembly, the method comprising: injecting a first precursor for forming an electrode layer onto a target nerve or into an area therearound in S110; placing a first photomask for formation of a target electrode layer structure on a skin, and performing a first light exposure process on the first precursor using the first photomask, thereby forming the electrode layer having the target electrode layer structure S120; injecting a second precursor for forming an energy harvesting layer onto the formed electrode layer in S130; and placing a second photomask for formation of a target energy harvesting layer pattern on the skin, and performing a second light exposure process on the second precursor using the second photomask, thereby forming the energy harvesting layer having the target energy harvesting layer pattern in S140.

In S110, the method includes injecting the first precursor for forming the electrode layer onto the target nerve or into the area around the target nerve.

In accordance with some embodiments of the method for manufacturing the stimulation system based on in-vivo self-assembly, the first precursor for forming the electrode layer includes: a photocurable polymer monomer; a core-shell particle including an upconversion nanoparticle as a core and an ultraviolet curing agent as a shell; and a conductive nanoparticle.

In S120, the method includes placing a first photomask for formation of a target electrode layer structure on a skin and performing a first light exposure process on the first precursor using the first photomask, thereby forming the electrode layer having the target electrode layer structure. After the first precursor for forming the electrode layer is injected in S110, the first photomask for formation of the target electrode layer structure is placed on a skin and the first light exposure process is performed on the first precursor using the first photomask, thereby forming the electrode layer having the target electrode layer structure in S120. In this case, the negative photo-lithography may be performed. In order to form the target electrode layer structure, the first photomask is attached onto the skin and then the infrared rays are irradiated. Thus, only the portion of the first precursor that has received the infrared rays is cured, while the other portion thereof that has not received the infrared rays and thus has not been cured is washed away by body fluids, so that the electrode layer having the desired structure may be patterned and shaped in the body without the surgery. The conductive nanoparticles are embedded in the electrode layer.

In S130, the method includes injecting the second precursor for forming the energy harvesting layer onto the formed electrode layer. The injection of the second precursor for forming the energy harvesting layer is performed on the electrode layer having the target electrode pattern.

In accordance with some embodiments of the method for manufacturing the stimulation system based on in-vivo self-assembly, the second precursor for forming the energy harvesting layer includes: a photocurable polymer monomer; a core-shell particle including an upconversion nanoparticle as a core and an ultraviolet curing agent as a shell; and a material for forming a piezoelectric layer.

In accordance with some embodiments of the method for manufacturing the stimulation system based on in-vivo self-assembly, the material for forming the piezoelectric layer includes a piezoelectric material or an ionic material.

In S140, the method includes placing a second photomask for formation of a target energy harvesting layer pattern on the skin; and performing a second light exposure process on the second precursor using the second photomask, thereby forming the energy harvesting layer having the target energy harvesting layer pattern. In order to form the desired energy harvesting layer pattern, the second photomask is attached onto the skin and then the infrared rays are irradiated thereto. Thus, only the portion of the second precursor that has received the infrared rays is cured, while the other portion thereof that has not received the infrared rays and thus has not been cured is washed away by body fluids, so that the energy harvesting layer having the desired pattern may be formed in the body without the surgical operation. The negative photo-lithography may be performed using the second photomask. The piezoelectric material or the ionic material is embedded in the energy harvesting layer.

The infrared light used in S120 or S140 has a wavelength of about 850 to 980 nm, and the light exposure time duration varies depending on the intensity of the infrared light and may be in a range of about several tens of seconds to 5 minutes.

Hereinafter, more specific examples and experimental examples will be described. However, the following examples are only some implementations of the present disclosure, and the scope of the present disclosure is not limited to the following examples.

### Example 1

The first precursor for forming the electrode layer includes: a photocurable polymer monomer; a core-shell particle including an upconversion nanoparticle as a core and an ultraviolet curing agent as a shell; and a conductive nanoparticle. The second precursor for forming the energy harvesting layer includes: a photocurable polymer monomer; a core-shell particle including an upconversion nanoparticle as a core and an ultraviolet curing agent as a shell; and a material for forming a piezoelectric layer.

The infrared (IR) curing photoinitiator includes the upconversion nanoparticle (UCNP) material and the ultraviolet curing agent which constitute the core-shell structure. In this regard, the UCNP is based on a phenomenon in which two or more low energy photons interact with each other into one high energy photon.

The order of manufacturing the neurostimulation system in the negative photo-lithography using the infrared curing photoinitiator is as follows. (1) First, the infrared light stimulus is absorbed by the UCNP material and converted into high energy photons, (2) the high energy photons emitted from the UCNP are transferred to the ultraviolet curing agent, and (3) the ultraviolet curing agent having absorbed the high energy photons cures the photocurable polymer monomer existing in the vicinity thereof.

The neural stimulation system introduced in the present disclosure comprises: 1) the energy harvesting layer for generating energy in the body in response to a mechanical stimulus applied thereto; and 2) the electrode layer for transferring the generated energy to a nerve tissue.
1) The energy harvesting layer may have the piezoelectric material (Barium Titanate, Zinc oxide, etc.) or the ionic material (NaCl, KCl, ionic liquid, etc.) embedded in the precursor material so as to generate an electric signal in response to the mechanical stimulus applied thereto.
2) The electrode layer may have a conductive nanofiller (metal nanoparticle, metal nanowire, metal flake, carbon, etc.) embedded in the precursor material.

After the first precursor constituting the electrode layer was first injected onto the target nerve, a cuff electrode for transmitting an electrical signal to the target nerve was manufactured by performing infrared patterning on the first precursor. Thereafter, the second precursor for forming the energy harvesting layer was injected onto the area where the cuff electrode has been manufactured and then subjected to the infrared patterning to form a self electricity generation layer in the body. In this manner, the neurostimulation system having the desired structure may be freely configured based on the characteristics of the neuropathy and the target nerve.

Referring to an embodiment of FIG. 2, \textcircled{1} An electrode layer precursor injection process: a process of injecting a first precursor material in which the photocurable polymer monomer, the UCNP/ultraviolet curing agent core-shell particle and the conductive nanofiller are mixed with each other into the body.
\textcircled{2} An electrode patterning process: a process of attaching the first photomask on the skin to form a desired structure, curing only a portion of the first precursor that has received the infrared light when the first precursor is exposed to the infrared light, and washing off a portion thereof that has not received the infrared light and thus has not been cured with the body fluid, thereby forming the electrode having the desired structure in the body without surgery
\textcircled{3} An energy harvesting precursor injecting process: a process of injecting a second precursor material in which the photocurable polymer monomer and the UCNP/ultraviolet curing agent core-shell particle are mixed each other onto the patterned electrode.
\textcircled{3} An energy harvesting layer patterning process: the second photomask is attached onto the skin again and the infrared light is irradiated to the second precursor such that only the portion of the second precursor that has received the light and has been cured, and the portion that has not been irradiated with infrared light and thus has not been cured is washed away with the body fluid, such that the energy harvesting layer is patterned in the body without surgery.

In this regard, a mixture in which poly(ethylene glycol) diacrylate (PEGDA), Chitosan, and Sodium alginate were mixed with each other was used as the polymer monomer, the upconversion nanoparticles were nanoparticles having a core-shell structure of NaYF₄:Yb/Tm@NaYF₄, and LAP (Lithium phenyl-2,4,6-trimethylbenzoylphosphinate) was used as the ultraviolet curing agent. Molybdenum (Mo) nanoparticles were used as the conductive nanoparticles. In the in vitro experiment, the first precursor was injected into between an issue-mimicking material and a PDMS layer with a syringe, and the photomask was placed on the PDMS and the infrared light was irradiated thereto. Thus, the electrode layer was patterned. In the in-vivo experiment, the precursor was injected into an area under the skin layer with a syringe, the photomask was placed on the skin, and the infrared rays were irradiated thereto. Thus, the electrode layer was patterned. The material for forming the energy harvesting layer is basically the same as the material for forming the electrode layer. However, the conductive nanoparticles were additionally added to the electrode layer material to increase conductivity, while chitosan and sodium alginate were not added to the energy harvesting layer. In this case, the piezoelectric energy generation layer was formed by including ions into the second precursor using a piezo-ionic method. In this regard, there is no sodium ion (Na⁺) or the like, the conductivity of the energy harvesting layer is relatively smaller than that of the electrode layer. The energy harvesting layer generates the electrical signal based on thermodynamic ion migration in response to the external mechanical stimulus applied thereto. The generated electrical signal is transmitted to the nerve through the electrode layer. For the formation of the energy harvesting layer, the second precursor was also injected into between the tissue-mimicking material and the PDMS layer with the syringe. In the in-vivo environment, the first precursor was injected, with the syringe, into the area under the skin layer. Similarly, the second photomask was placed on the skin and the infrared rays were irradiated to the second precursor. Thus, the energy harvesting layer was patterned. The infrared rays may have a wavelength of about 850 to 980 nm. The exposure time duration varies depending on the intensity of the infrared rays and may be in a range of about tens of seconds to 5 minutes.

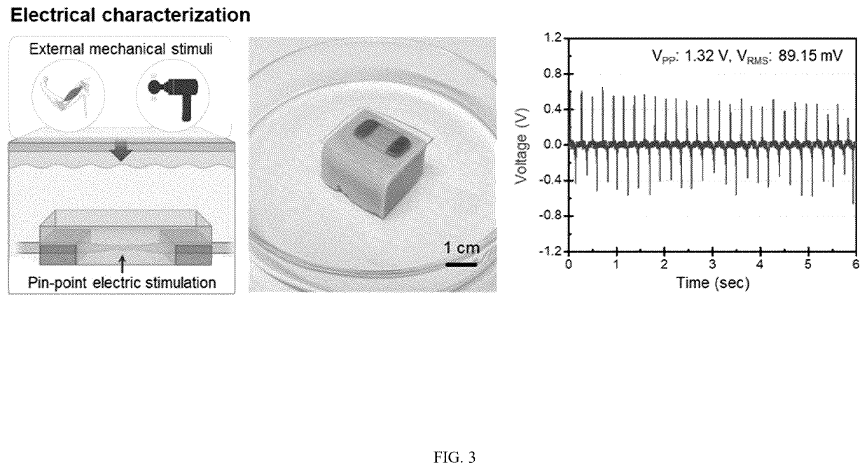

Energy generation in the body according to external mechanical stimulation applied to the system manufactured in this way was measured. FIG. 3 shows a result of measuring in-vivo energy generation according to external mechanical stimulation applied to a system according to an embodiment of the present disclosure. As shown in FIG. 3, the system has been formed between the tissue mimetic material and the PDMS, and then, the external mechanical stimulus is applied thereto using a pushing machine or the like. Then, an electric signal generated from the system is measured using an oscilloscope to measure a voltage graph as shown in the right side of FIG. 3.

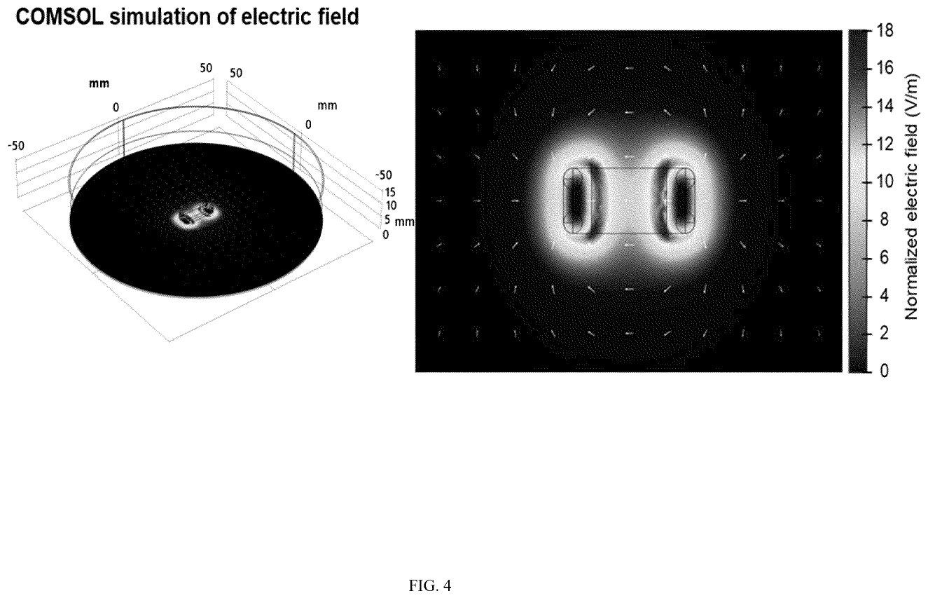

FIG. 4 is a diagram observing the in-vivo energy generation and electric field generation under the external mechanical stimulation. A result of simulating an electric field generated from the system using a finite element analysis (FEM simulation) based on the experiment of FIG. 3 is shown in FIG. 4.

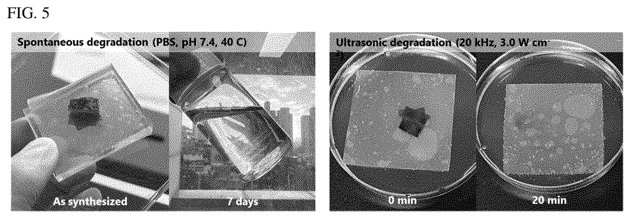

FIG. 5 is a diagram checking the biodegradability of the system. In order to check whether the system is biodegradable, it was identified that the system was immersed in PBS buffer solution (pH 7.4, 40 degrees C.), and then the system was decomposed and removed away after 7 days. In addition, it was identified whether the biodegradation rate could be promoted via ultrasonic application. It was identified that when the ultrasonic waves with a frequency of 20 kHz and an intensity of 3.0 W cm⁻² was applied to the system, the system was decomposed and removed away after about 20 minutes.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A non-surgical neural stimulation system based on in-vivo self-assembly, the system comprising:
an electrode layer formed by injecting a first precursor for forming the electrode layer onto a target nerve or into an area therearound and performing a first light exposure process on the first precursor using a first photomask; and
an energy harvesting layer formed by injecting a second precursor for forming the energy harvesting layer onto the electrode layer and performing a second light exposure process on the second precursor using a second photomask.

2. The non-surgical neural stimulation system based on in-vivo self-assembly of claim 1, wherein the first precursor for forming the electrode layer includes:
a photocurable polymer monomer;
a core-shell particle including an upconversion nanoparticle as a core and an ultraviolet curing agent as a shell; and
a conductive nanoparticle.

3. The non-surgical neural stimulation system based on in-vivo self-assembly of claim 2, wherein the conductive nanoparticle is embedded in the electrode layer.

4. The non-surgical neural stimulation system based on in-vivo self-assembly of claim 1, wherein the second precursor for forming the energy harvesting layer includes:
a photocurable polymer monomer;
a core-shell particle including an upconversion nanoparticle as a core and an ultraviolet curing agent as a shell; and
a material for forming a piezoelectric layer.

5. The non-surgical neural stimulation system based on in-vivo self-assembly of claim 4, wherein the material for forming the piezoelectric layer includes a piezoelectric material or an ionic material.

6. The non-surgical neural stimulation system based on in-vivo self-assembly of claim 5, wherein the piezoelectric material or the ionic material is embedded in the energy harvesting layer.

7. The non-surgical neural stimulation system based on in-vivo self-assembly of claim 1, wherein each of the first and second photomasks enables negative photo-lithography.

8. A method for manufacturing a non-surgical neural stimulation system based on in-vivo self-assembly, the method comprising:
injecting a first precursor for forming an electrode layer onto a target nerve or into an area therearound;
placing a first photomask for formation of a target electrode layer structure on a skin;
performing a first light exposure process on the first precursor using the first photomask, thereby forming the electrode layer having the target electrode layer structure;
injecting a second precursor for forming an energy harvesting layer onto the formed electrode layer;
placing a second photomask for formation of a target energy harvesting layer pattern on the skin; and
performing a second light exposure process on the second precursor using the second photomask, thereby forming the energy harvesting layer having the target energy harvesting layer pattern.

9. The method for manufacturing the stimulation system based on in-vivo self-assembly of claim 8, wherein the first precursor for forming the electrode layer includes:
a photocurable polymer monomer;
a core-shell particle including an upconversion nanoparticle as a core and an ultraviolet curing agent as a shell; and
a conductive nanoparticle.

10. The method for manufacturing the stimulation system based on in-vivo self-assembly of claim 9, wherein the conductive nanoparticle is embedded in the electrode layer.

11. The method for manufacturing the stimulation system based on in-vivo self-assembly of claim 8, wherein the second precursor for forming the energy harvesting layer includes:
a photocurable polymer monomer;
a core-shell particle including an upconversion nanoparticle as a core and an ultraviolet curing agent as a shell; and
a material for forming a piezoelectric layer.

12. The method for manufacturing the stimulation system based on in-vivo self-assembly of claim 11, wherein the material for forming the piezoelectric layer includes a piezoelectric material or an ionic material.

13. The method for manufacturing the stimulation system based on in-vivo self-assembly of claim 12, wherein the piezoelectric material or the ionic material is embedded in the energy harvesting layer.

14. The method for manufacturing the stimulation system based on in-vivo self-assembly of claim 8, wherein each of the first and second photomasks enables negative photo-lithography.
